(19) United States
(12) Patent Application Publication
(10) Pub. No.: US 2024/0390461 A1
(43) Pub. Date: Nov. 28, 2024

(54) TREATMENT WITH TRANSFORMING GROWTH FACTOR-BETA PROTEINS TO PREVENT AND TREAT HEARING LOSS

(71) Applicant: Benjamin Button, LLC, Boca Raton, FL (US)

(72) Inventor: Christian PAULUS, Kalispell, MT (US)

(21) Appl. No.: 18/674,340

(22) Filed: May 24, 2024

Related U.S. Application Data
(60) Provisional application No. 63/468,698, filed on May 24, 2023.

(51) Int. Cl. A61K 38/18; A61P 27/16

(52) U.S. Cl. CPC A61K 38/1841; A61P 27/16

(57) ABSTRACT

Compositions and methods employ a Transforming Growth Factor-beta superfamily protein to treat a neural disorder characterized by a lack of differentiated functional sensory and neural cells in the inner ear. The TGF-beta superfamily protein GDF-11 is employed in the treatment of diseases in which auditory differentiated cells provide for a repair mechanism to the disease. GDF-11 is employed to increase production of sensory cells of the cochlear epithelium as well as stimulate sensory receptors in the setting of hearing loss.

### RELATED APPLICATION

This application claims priority to U.S. provisional patent application Ser. No. 63/468,698, filed on May 24, 2023, and entitled Treatment With Transforming Growth Factor Beta proteins To Prevent and Treat hearing Loss, the contents of which are herein incorporated by reference.

### BACKGROUND OF THE INVENTION

The present invention is directed to transforming growth factor-beta (TGF-beta) superfamily proteins, and more particularly to TGF-beta proteins as they relate to the prevention and treatment of hearing loss.

Many neural disorders eventually result in an irreversible loss of function of the affected organ, and typically only few treatment options remain available for such diseases, primarily in the realm of disease management, rather than reversal. For example, sensorineural hearing loss cannot currently be reversed as cochlear hair cells or cochlear epithelial cells cannot yet be regenerated. The most advanced available treatments cannot improve hearing loss without the use of devices such as hearing aids or implanted electrical stimulators. While these devices can provide amplification of the remaining hearing, the technologies do not address the actual underlying hearing loss disease which continues to worsen, often requiring more advanced and expensive hearing devices. At the same time, patients with early-stage hearing loss are not candidates for these devices and thus have no current options to prevent the progression of the disease or have options how to optimize the remaining hearing function.

Therefore, while there are some devices, compositions, and methods for improving neural function are known in the art, all or almost all of them suffer from one or more disadvantages. Consequently, there is still a need to provide improved compositions and methods to restore neural, and especially auditory function.

### SUMMARY OF THE INVENTION

The compositions and methods disclosed herein relate to the findings that transforming growth factor-beta (TGF-beta) superfamily proteins may be used to prevent, and in certain embodiments treat, hearing loss and related impairments. For example, the methods and compositions disclosed herein for preventing or reducing hearing loss in a subject cause a corresponding reduction in the loss of speech impairments and cognitive function. Also disclosed herein are novel compositions and methods for preventing, and in certain instances treating, hearing loss. In certain aspects, the compositions and methods disclosed herein can be used to prevent hearing loss in individuals due to exposure to noise pollution, medication toxicity, prior ear infections, ear trauma or advancing age.

The transforming growth factor-beta (TGF-beta) superfamily protein treatment apply to sensorineural, conductive and mixed hearing loss.

Methods of reducing and/or treating hearing loss in a subject in need thereof, such methods comprising a step of administering to the subject a composition which increases protein levels related to the Transforming Growth Factor-beta (TGF-beta) superfamily, such as Growth Differentiation Factor 11 (e.g., GDF11) polypeptides in the subject and thereby reduces or treats sensorineural hearing loss.

The administration of a composition comprising human GDF11 polypeptide, an GDF11 analog or a functional fragment or variant thereof, to the subject hearing loss in a subject helps prevents, inhibit and/or treat speech impediments and loss of cognition.

It is further contemplated that increasing levels of the transforming growth factor-beta (TGF-beta) superfamily proteins increases the production of auditory sensory cells, as well as the sensitivity of sensory neurons to sound transmission.

According to one embodiment, the present application is directed to a method of treating a hearing loss condition comprising administering to a subject a composition which increases the level of a GDF11 polypeptide in the subject. The hearing loss condition can be age related hearing loss, noise pollution, medication toxicity, prior ear infections, ear trauma, and the like. The level of the GDF11 polypeptide in the subject to treat the hearing loss condition can be a level of GDF11 in the circulation of the subject. The composition can include a GDF11 polypeptide or a prodrug or an analog. The composition can be administered via a route such as intravenously, subcutaneously, intramuscularly, orally, sublingually, trans dermally, or rectally.

The level of the GDF11 in the subject can be increased by at least 10%. Further, at least one of the GDF11 and the GDF11 analog is a recombinant protein. The GDF11 can include BMP11 and the method can include treating the subject with 30 picograms of the BMP11. Optionally, the method can include treating the subject daily for three months or 6 months in duration, or treating the subject with between about 10 and about 100 picograms of the GDF11. Optionally, the subject can be treated with between about 20 and about 40 picograms of the GDF11, or with 50 picograms of the BMP11.

The present invention is also directed to a pharmaceutical composition having an isolated GDF11 polypeptide and a pharmaceutically acceptable carrier. The present invention an also be directed to the use of at least one of a GDF-11 or a GDF-11 analog in the manufacture of a drug for treatment of an auditory neural disorder. The auditory neural disorder can be selected from the group consisting of sensorineural hearing loss and conductive hearing loss.

Various objects, features, aspects and advantages of the present invention will become more apparent from the following detailed description of preferred embodiments of the invention.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a graph depicting composite hearing scores in relation to time in an individual treated with the transforming growth factor-beta (TGF-beta) superfamily protein GDF11. Composite hearing scores can be seen rising in response to the treatment.

FIG. 2 is a table depicting audiogram hearing tests at sequential dates from 250-8,000 Hz frequencies with correlating decibel hearing levels related to the GDF11 treatments depicted in FIG. 1. Hearing decibel levels are shown to lower over time, indicating the subjects improved ability to detect sound at all frequencies.

### DETAILED DESCRIPTION

Hearing loss is known to be a significant sensory impairment, afflicting approximately 5% of the world's population in an often-disabling manner. Hearing loss results in many consequences including loss of cognition and the development of dementia, speech impediments, social stigma and general loss of quality of life.

The compositions and methods disclosed herein are useful for the prevention or treatment of hearing loss. The specific hearing loss may be caused by many factors, including hereditary versions, disease, medication toxicity, noise exposure or by trauma.

Disclosed herein are compositions and methods that relate to the finding that TGF-beta superfamily protein GDF11 can prevent and treat hearing loss. In particular, the methods and compositions described herein relate to increasing the level of GDF11 polypeptide in a subject, thereby treating or preventing the development of hearing loss.

According to numerous studies, even in the context of chronic and severe hearing loss, viable progenitor cells remain in the auditory epithelium. The progenitor cells are considered to be dormant and hence inactive. The present inventor believes that GDF11 can be employed to stimulate and activate the residual progenitor cells resulting in progenitor cell differentiation into cells of the primary auditory pathway, including inner and outer hair cells of the vestibulo-cochlear epithelium, neurons and glia of the spiral acoustic ganglion, and the vestibulo-cochlear nerve. Additionally, it is contemplated herein that GDF11 can have a direct sensory receptor stimulant effect in the organ of corti.

As used herein, the term “GDF11” refers to Growth and Differentiation Factor 11 (NCBI Gene ID No: 10220), which is a member of the Transforming Growth Factor-beta (TGF-beta) superfamily of growth factors. The terms can also refer to fragments or variants of GDF11 (e.g., non-naturally occurring variants) that maintain at least 50% of the beneficial effects. In certain aspects, the GDF11 proteins or polypeptides disclosed herein have been modified (e.g., modified to extend the therapeutic activity of such GDF11 protein or polypeptide). In certain aspects, the GDF11 proteins or polypeptides have been modified such that they are non-naturally occurring variants of GDF11 (e.g., functional variants of human GDF11). Conservative substitution variants that maintain the effects of wild type GDF11 will include a conservative substitution as defined herein. The identification of amino acids most likely to be tolerant of conservative substitution while maintaining at least 50% of the activity of the wild type GDF11 is guided by, for example, sequence alignment with GDF11 homologs or paralogs from other species.

As used herein, “GDF11” can include the human precursor polypeptide (SEQ ID NO: 1, NCBI Ref Seq: NP 005802); the human pro-peptide (SEQ ID NO: 2); the human N-terminal polypeptide (SEQ ID NO: 4), and the human mature (SEQ ID NO: 3) forms of GDF11 as well as homologs from other species, including but not limited to bovine, dog, cat, chicken, murine, rat, porcine, bovine, turkey, horse, fish, baboon and other primates.

“Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when refining to a gene product and fragments thereof.

In certain aspects, the GDF11 can be modified (e.g., modified to extend its therapeutic activity). As used herein, the term “modified” generally refers to changing GDF11 to impart one or more properties or to alter the activity of GDF11 in a selective manner, so as to cause GDF11 to exert a desired physiological or biological effect. It should be noted that in certain embodiments, modification includes coupling GDF11 to one or more secondary compounds or molecules (e. g., coupling GDF11 or a fragment or variant thereof to a fusion protein or to one or more polymers).

Embodiments of the technology of the present invention described herein are based on the discovery that the level of GDF11 in the blood of humans decreases with age and this decrease in GDF11 level is associated with a decline in human organ function.

The methods and compositions described herein generally relate to increasing the level of GDF11 (e.g., GDF11 polypeptide) in a subject to treat, prevent, inhibit, or reverse sensorineural hearing loss.

In some embodiments of the present invention, the subject has or has been diagnosed with a condition selected from the group consisting of sensorineural hearing loss, conductive hearing loss, or mixed hearing loss consisting of some components of each.

In one aspect of this disclosure, the technology described herein relates to a pharmaceutical composition comprising an isolated GDF11 polypeptide and a pharmaceutically acceptable carrier. Compound, or mixture.

In certain aspects, the methods disclosed herein comprise a step of administering to a subject a composition which increases GDF11 precursor-peptide (pro-drug), polypeptide in the subject (e.g., administering a human GDF11 polypeptide or a functional fragment or variant thereof). For example, in some embodiments, such a composition comprises a GDF11 polypeptide (e.g., human recombinant GDF11) or a functional fragment or variant thereof (e.g., a non-naturally occurring functional variant of human GDF11). In some embodiments, the GDF11 polypeptide or a functional fragment or variant thereof is recombinantly prepared. In certain aspects, the composition stimulates the endogenous expression and/or production of GDF11 in the subject in any of the foregoing embodiments.

In certain embodiments, the GDF11 polypeptide may be modified (e. g., pegylation, post-translational modifications (PTMs), cleavage, folding and conformational changes) mutated or coupled to another substance to impart one or more desired characteristics to the polypeptide (e.g., to extend its therapeutic activity).

In other embodiments, the compound is a GDF11 analog. With respect to suitable TGF-beta superfamily proteins contemplated herein, it should be appreciated that while GDF11 is a preferred compound, numerous alternative compounds are also be deemed suitable so long as such compounds interact with one or more components in a signaling pathway in a manner that is functionally associated with selected members of the TGF-beta superfamily. Thus, and viewed from one perspective, chemically (e.g., pegylated, acylated, etc.) and/or biologically (e.g., mutated, truncated, fused, enzymatically modified, etc.) modified versions of GDF11 may be suitable, as well as GDF11 analogs from a species other than human. Viewed from another perspective, suitable GDF11 alternatives also include those molecules that yield at least a moderate signal response in a GDF11 associated pathway.

Viewed from yet another perspective, all molecules other than GDF11 may also be suitable that bind to receptor/binding sites to which GDF-11 is known to bind. Still further, it should be noted that (typically synthetic) Small molecule GDF-11 agonists are also contemplated for use herein.

The compositions contemplated by the present invention can be administered to a subject by any suitable routes of administration. For example, in certain aspects the compositions disclosed herein (e.g., a composition comprising recombinantly-prepared human GDF11 protein) are administered to a subject via a route of administration selected from the group consisting of oral, sublingual, transdermal, enterally, intravenously, subcutaneously, intramuscularly and intrathecally.

In some embodiments, a GDF11 variant as described herein can be administered by controlled- or delayed-release mechanisms.

It is generally contemplated that the compounds according to the present invention can be prepared in a formulation for parenteral use (e.g., intravenous (IV), intramuscular (IM), subcutaneous (SC), or intradermal (ID) injection), and especially contemplated parenteral formulations can be liquid formulations for injection. Therefore, appropriate formulations can generally include a pharmaceutically acceptable solvent (e.g., sterile isotonic aqueous or non-aqueous solution), and can be prepared as a dispersion, Suspension, or emulsion. Examples of suitable aqueous and non-aqueous carriers which may be employed in the pharmaceutical compositions include water, ethanol, polyols (e.g., glycerol, propylene glycol, polyethylene glycol, etc.), and suitable mixtures thereof, vegetable oils, and injectable organic esters, such as ethyl oleate.

It is further preferred that contemplated formulations are in unit dosage form. It is still further preferred that the amount of the contemplated compound (active ingredient) that is combined with a carrier to form a unit dosage form can be the amount that produces a therapeutic effect. Depending on the formulation, the percentage (% weight) of the active ingredient can typically range from about 0.001 percent to about ninety-nine percent of the total weight, more preferably from about 0.01 percent to about 70 percent, and most preferably from about 0.01 percent to about 50 percent.

According to another embodiment, the GDF11 can be administered to the subject in a dosage range of between about 10 to about 100 picograms, and between about 20 and about 60 picograms. The subject can be treated daily and for a duration of between about 1 month and about 12 months, and between about 3 months and about 6 months.

In certain aspects, an effective amount of the composition is administered to the subject. Generally, the dosage can vary with the age, condition, and sex of the patient and can be readily determined by one of skill in the art based on the teachings herein. The dosage can also be adjusted by the individual physician in the event of any complication. For example, in certain aspects, the GDF11 polypeptide is administered to the subject at a dose (e. g., a daily dose) of about 1 picogram/kg to 1 mg/kg depending on the route of administration, disease duration and severity and other factors.

The dosage ranges for the agent depends upon the potency and are amounts large enough to produce the desired effect. The dosage should not be so large as to cause unacceptable adverse side effects. Generally, the dosage can vary with the age, weight, gender, type and severity of hearing loss, and can be determined by one of skill in the art. The dosage can also be adjusted by the individual physician in the event of any complication.

Typically, the dosage can range from 1 picogram/kg body weight to 0.5 mg/kg body weight depending on the route of administration, patient weight, degree of severity of the hearing loss among others. In one embodiment, the dose range is from 5 picogram/kg body weight to 5 nanogram/kg body weight.

In some embodiments, the level of GDF11 protein in the subject is increased by at least 100%, at least 90%, at least 80%, at least 70% or at least 50%.

In certain embodiments, the compositions disclosed herein may be formulated for multiple daily doses, daily, weekly, bi-weekly, monthly, bi-monthly, quarterly, biannual, semi
annual or annual administration.

The duration of treatment depends upon the subject's clinical progress and responsiveness to therapy, as well as based on other biological and behavioral indications.

Where the GDF11 polypeptide apparently diminishes with age in affected individuals, it is expected that long-term therapy may be required to establish and maintain the benefit of GDF11-based treatment, e.g. for age-related hearing loss.

It is generally preferred that the daily dose of contemplated compounds can typically correspond to the amount of the compound which is the lowest dose effective to produce a desired therapeutic effect.

In the treatment and/or prophylaxis of such diseases, it is generally preferred that the compounds or compositions according to the inventive subject matter are formulated in a pharmaceutically acceptable manner. Suitable formulations preferably include liquid preparations for injection into the anterior and/or posterior chamber of the eye, or for injection into the semicircular canals, cochlea, and/or bony labyrinth of the temporal bone. Alternatively, or additionally, implantable carriers (e.g., biodegradable/dissolving) may be formulated, such that the carrier comprises therapeutically effective amounts of the compound or composition, and that the carrier can release the compound or composition in a controlled and predetermined manner. Among other Suitable carriers, the release may be time-dependent and/or initiated by irradiation with light of one or more wavelengths.

Efficacy of an agent can be determined by assessing auditory measurements, such as the use of audiograms, word recognition testing, and the like. Methods of measuring these indicators are known to those of skill in the art and/or described herein.

The GDF11 polypeptide may be native and isolated from a biological source, or recombinant or produced in situ in neural tissue (e.g., via transfection).

In some embodiments, the GDF11 polypeptide is combined with other substances such as other peptides of polypeptides in order to add further potency or extend the spectrum of activity.

In some embodiments, the methods further comprise administering the pharmaceutical composition described herein along with one or more additional agents, biologics, drugs, or treatments beneficial to a subject suffering from age related hearing loss.

In some aspects of any of the foregoing embodiments, the subject is a mammal. In some aspects of any of the foregoing embodiments, the subject is a human, either infant, child or adult.

A subject can be one who has been previously diagnosed with or identified as suffering from or having hearing loss or one or more complications related to such a condition, and optionally, but need not have already undergone treatment for a condition or the one or more complications related to the condition.

Alternatively, a subject can also be one who has not been previously diagnosed as having hearing loss in need of treatment or one or more complications related to such a condition. Rather, the subject can include one who exhibits one or more risk factors for a hearing loss or one or more complications related to hearing loss. A “subject in need” of treatment can be a subject having hearing loss, diagnosed as having hearing loss, or at increased risk of developing hearing loss relative to a given reference population. The above discussed, and many other features and attendant advantages of the present inventions will become better understood by reference to the following detailed description of the invention.

The practice of the present invention can typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art.

In some embodiments, a GDF11 variant as described herein can be a pharmaceutically acceptable solvate. The term “solvate” refers to a peptide as described herein in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried under ambient conditions.

Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition will depend on the nature of the disorder or condition and can be determined by standard clinical techniques.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in each order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure considering the teachings herein. However, the scope of the present invention is defined by the appended claims.

Potential routes of administration of GDF11 include but are not limited to sublingual (e.g., liquid, film strips, immediate and extended-release capsule) administration, other oral administration techniques (e.g., capsules, pill, liquid), intramuscular, intravenous and transdermal.

The potential dosing intervals can include administering a therapeutically effective amount of the GDF11 to the subject either daily, twice daily, bi-weekly, weekly, and other dosing intervals.

The determination of suitable dosing in terms of quantities of GDF11 can depend on the gender, age, body mass, type of hearing loss, during of hearing loss, and severity of hearing loss, among other factors, of the subject or patient.

The duration of treatment can depend primarily on the type and severity of hearing loss.

Hearing loss or a hearing loss condition is a sensory impairment condition that is characterized by a partial or total inability to perceive sounds within a normal range of human hearing. It is a condition that affects a person's ability to detect, interpret, or understand sounds and speech. More specifically, hearing loss is a sensory disorder characterized by a diminished ability to perceive auditory stimuli, resulting in a reduced or complete loss of hearing sensitivity. The hearing loss can occur as a result of various factors, including genetic predisposition, aging, prolonged exposure to loud noises or noise pollution, certain medical conditions, infections, trauma, or medications (e.g., medication toxicity). Hearing loss can be categorized based on its severity, ranging from mild to profound, and can affect one or both ears. The degree and type of hearing loss may vary, leading to difficulties in communication, speech comprehension, social interactions, and overall quality of life.

As used herein, the term “auditory neural disorder” or “neural hearing disorder” refers to a condition that affects the neural pathways or structures involved in the transmission and processing of sound signals within the auditory system and can be characterized by abnormalities or impairments in the way the auditory nerves, brainstem, or central auditory pathway function, resulting in difficulties in perceiving, processing, or understanding auditory information. Specifically, the auditory neural disorder is a condition characterized by abnormalities or dysfunctions in the neural pathways responsible for the transmission and processing of auditory information. It typically involves impairments in the auditory nerves, brainstem, or central auditory pathways that affect the accurate perception and interpretation of sound signals. Auditory neural disorders can result in various auditory processing difficulties, such as difficulties in speech discrimination, sound localization, auditory sequencing, and auditory memory. These disorders can be present from birth (congenital) or acquired due to factors such as genetic abnormalities, trauma (e.g., sensorineural hearing loss), infections, certain medical conditions, or as a side effect of medications. Treatment options for auditory neural disorders may include auditory training, assistive listening devices, cochlear implants, or other forms of auditory rehabilitation, depending on the specific nature and severity of the disorder. The disorder can include sensorineural hearing loss and conductive hearing loss. The conductive hearing loss can be a type of hearing loss that occurs when there is a problem with the conduction of sound through the outer or middle ear and is characterized by a reduction in the ability to hear sounds, especially at lower volumes. The conductive hearing loss can include impaired sound conduction where hearing loss occurs when sound waves are unable to pass efficiently through the outer ear, ear canal, or middle ear structures, including the eardrum and the tiny bones (ossicles) known as the malleus, incus, and stapes. This can result in a decrease in the volume or intensity of sound reaching the inner ear.

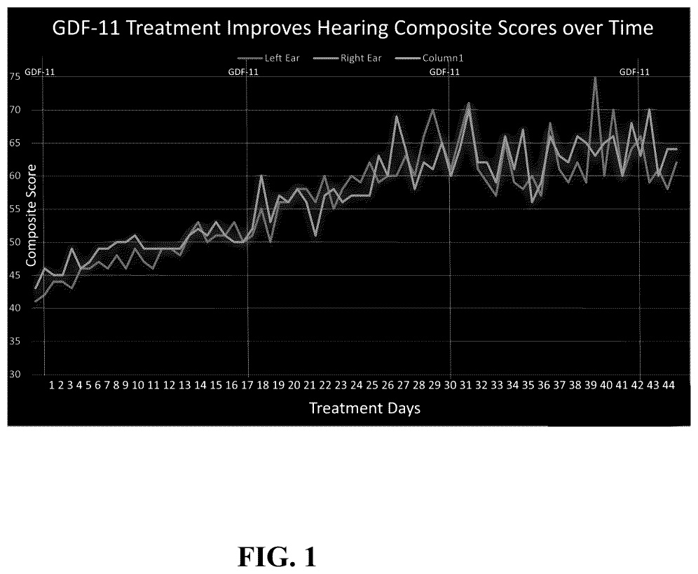

FIG. 1 is a graph depicting composite hearing scores in relation to time in an individual treated with a transforming growth factor-beta (TGF-beta) superfamily protein GDF11. As clearly shown in the graph, the composite hearing scores can be seen rising over time in response to treating the patient GDF11.

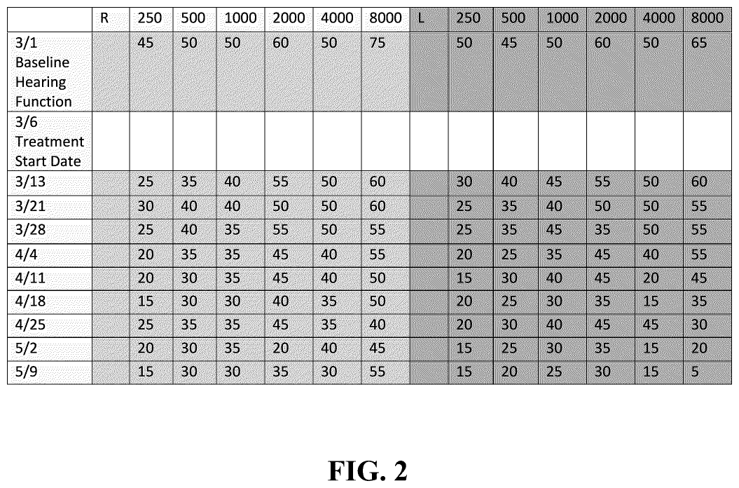

FIG. 2 is a table depicting audiogram hearing tests at sequential dates from 250-8,000 Hz frequencies with correlating decibel hearing levels in both the left and right ears, and related to the GDF11 treatments depicted in FIG. 1. The table depicts and shows a baseline hearing function for the subject as tested. The table also shows the hearing function during treatment at different dates. The hearing decibel levels are shown to lower over time, indicating that the subjects improved ability to detect sound at all frequencies.

### EXAMPLES

The following are working examples that demonstrate the utility and feasibility of the present invention of the present invention.

### Example 1

A 43-year-old female patient with a 5-year history of moderate age-related sensorineural hearing loss was treated with a dose of 30 picograms of sublingual GDF11 (BMP-11) daily for a 3-month duration. Serial formal audiograms showed initial improvements in high-frequency hearing capacity after 4 weeks of treatment. Further improvements were noted throughout the remainder of the 3-months treatment period. Final audiograms documented a total of 27% improvement in the Speech Recognition Threshold (SLR).

### Example 2

A 69-year-old male patient with a 10-year history of severe sensorineural hearing loss attributed to significant and long-standing noise exposure was initially treated with a dose of 50 picograms of daily sublingual GDF-11 (e.g., Bone Morphogenetic Protein 11 (BMP11)) for a 6-month duration. Serial audiograms documented an initial improvement of 31% in the Speech Recognition Threshold (SLR) at the 6 months mark. Dose escalation of GDF11 (BMP11) to 400 picograms daily did not result in further gains.

### Example 3

A 50-year-old female patient with moderate sensorineural hearing loss of unknown etiology and recent hearing aid dependency was treated with a daily dose of 30 picograms of sublingual GDF-11 (BMP-11) for a 6-month duration. Daily audiograms showed a measurable improvement in the composite hearing score after 3 weeks of treatment. The patient was not in need of hearing aids for the majority of the week according to daily hearing aid use diaries.

The following are prophetic examples that demonstrate the utility and feasibility of the present invention.

### Example 4

A 50-year-old male with a 8-year history of antibiotic induced moderate sensorineural hearing loss and impending need for hearing aids would be treated with a dose of 40-60 picograms daily of sublingual GDF11 (e.g., BMP11) for an initial period of 6 months. It is anticipated that serial audiograms and Speech Recognition Threshold (SLR) assessments will document a stabilization of the hearing loss with sufficient improvement to avoid the need for a hearing aid. Following the initial 6-months treatment period, a dose reduction of GDF11 (e.g., BMP11) to 10-20 picograms daily would be sufficient to maintain the initial benefit gained for the long term.

### Example 5

A 76-year-old female patient with a 31-year history of severe sensorineural hearing loss of uncertain etiology, has been hearing aid dependent for the last 24 years. Despite numerous adjustments in the hearing aid setting as well as upgrading the hearing aids to the most advanced models, her hearing loss is now in the profound range with very little benefit from the hearing aids. It is anticipated that an initial course of 30-50 picograms daily of sublingual GDF-11 (BMP-11) for a 3-months duration would result in an improvement in audiogram documented Speed Recognition Thresholds (SLR). This benefit is anticipated to further stabilize and maintain itself with a durational dose of 10-20 picograms of daily sublingual GDF11 (e.g., BMP11).

### Example 6

An 11-year-old child with congenital sensorineural hearing loss is a candidate for hearing aids. An initial daily dose of 20 picograms sublingually would be expected to avoid the initial need for a hearing aid. Subsequent daily doses of GDF-11 (BMP-11) in the 20-40 picogram range may maintain the initial hearing benefit and may offer further improvements.

I claim:

1. A method of treating a hearing loss condition, the method comprising administering to a subject a composition which increases the level of a GDF11 polypeptide in the subject.

2. The method of claim 1, wherein the hearing loss condition is selected from the group consisting of: Age related hearing loss, noise pollution, medication toxicity, prior ear infections, ear trauma.

3. The method of claim 2, wherein the level of the GDF11 polypeptide is a level of GDF11 in the circulation of the subject.

4. The method of claim 1, wherein the composition comprises a GDF11 polypeptide.

5. The method of claim 1, wherein the composition comprises a GDF-11 prodrug or analog.

6. The method of claim 1, wherein the composition is administered via a route selected from the group consisting of: intravenously, subcutaneously, intramuscularly, orally, sublingually, trans dermally, or rectally.

7. The method of claim 1, wherein the level of the GDF11 is increased by at least 10%.

8. The method of claim 5, wherein at least one of the GDF11 and the GDF11 analog is a recombinant protein.

9. The method of claim 1, wherein the GDF11 comprises BMP11.

10. The method of claim 9, further comprising treating the subject with 30 picograms of the BMP11.

11. The method of claim 10, further comprising treating the subject daily for three months.

12. The method of claim 11, comprising sublingually administering the BMP11 to the subject.

13. The method of claim 9, further comprising treating the subject with 50 picograms of the BMP11.

14. The method of claim 13, further comprising treating the subject daily for a 6-month duration.

15. The method of claim 1, further comprising treating the subject with between about 10 and about 100 picograms of the GDF11.

16. The method of claim 1, further comprising treating the subject with between about 20 and about 40 picograms of the GDF11

17. The method of claim 1, further comprising treating the subject with between about 40 and about 60 picograms of the GDF11

18. A pharmaceutical composition, comprising an isolated GDF11 polypeptide and a pharmaceutically acceptable carrier.

19. Use of at least one of a GDF-11 or a GDF-11 analog in the manufacture of a drug for treatment of an auditory neural disorder.

20. The use of claim 19, wherein the auditory neural disorder is selected from the group consisting of sensorineural hearing loss and conductive hearing loss.
